(19) United States
(12) Patent Application Publication
(10) Pub. No.: US 2010/0048689 A1
(43) Pub. Date: Feb. 25, 2010

(54) PROCESS OF OBTAINMENT OF SOY ISOFLAVONES

(76) Inventors: Kun Park Yong, Campinas - Sao Paulo (BR); Claudio Lima De Aguar, Londrina - Parana (BR); Maria Cristina Youn Lui, Campinas - Sao Paulo (BR)

Correspondence Address: LADAS & PARRY LLP, 224 SOUTH MICHIGAN AVENUE, SUITE 1600, CHICAGO, IL, 60604, US

(21) Appl. No.: 12/519,281

(22) PCT Filed: Dec. 14, 2006
(86) PCT No.: PCT/BR06/00277

§ 371 Date: Oct. 6, 2009

(51) Int. Cl. A61K 31/352; C12P 21/00; C12P 17/06; C12N 1/14; A61P 43/00; A23L 1/29

(52) U.S. Cl. 514/456; 435/71.2; 435/125; 435/256.1; 435/254.1; 426/61

(57) ABSTRACT

The present invention relates to the process for the recovery of conjugated isoflavones of residues and sub-products of food industries based on the use of soy and its derivatives. It also concerns of isoflavones obtained from food composition containing isoflavones and from the fungus genetically modified used in this process, Aspergillus oryzae ATCC 22786 (RIB 430). More specifically, the present invention concerns of a process of conversion of conjugated isoflavones, in the form of isoflavone malonate and acetates, in glucosylated isoflavones, which through fermentative and enzymatic processes are transformed into aglycone isoflavones. The products obtained in this process, pass to show a promising therapeutic as nutritive application as previously described, and may be used as a functional food or as a functional ingredient.

### FIELD OF INVENTION

The present invention relates to a process for the recovery of isoflavones in all forms, whether conjugated, such as malonate, acetate, glucosylated or free, such as aglycone isoflavone, from residues and sub-products of food industries which use soybean and its derivatives as raw-material, following an enzymatic hydrolysis process of all isoflavones extracted, converting them to their respective aglycone isoflavones.

### FUNDAMENTS OF THE INVENTION

A recent increase on the demand of the consumption of soy based food has promoted the greater demand for grain soybean in Brazil and in the World. Brazil is one the greatest grain soybean producer and is a great exporter of soy grains, flour and oil. However, the industrial use of soybean includes other derivative products, such as: concentrated and isolated soy protein, lecithin, isoflavones, non-fermented products (1. soy hydrosoluble extract, 2. tofu, 3. yuba, 4. okara) and fermented products (1. jiang or miso, 2. jiangyou or shoyu, 3. tempeh, 4. natto, 5. douche or hamanatto, 6. sufu).

Soy is a valorous source of chemical compounds which benefits human health, such as: oils, proteins and isoflavones.

A diet rich in soy isoflavones, mainly genistein is associated to a reduced incidence of vasomotor episodes. The daily mean supplementation of genistein is 50 mg/day. After supplementing the diet with soy isoflavones, studies show a reduction in total cholesterol and in LDL fraction. There was also an increase in bone mineral density after the ingestion of 90 mg of isoflavones during 6 months. Isoflavones may reduce the risk of the development of breast cancer. Data published confirm the excellent clinical efficacy of the diet supplementation with soy extract, particularly genistein, which has shown the relief of short-term symptoms of menopause and the long-term effects (ARENA, S et. al., 2002)

Soy contains a high concentration of isoflavones, which may show twelve different isomeric forms, namely: aglycones (daidzein, glycitein and genistein), glucosylated (daidzin, glycitin and genistin), malonyl conjugated (6″-O-malonyidaidizin, 6″-O-malonylglycitin and 6″-O-malonylgenistine) and acetyl conjugated (6″-O-acetyldaidzin, 6″-O-acetylglycitin and 6″-O-acetylgenistin) (KLUS & BARS, 1998).

Isoflavones on soy are basically shown under glucosylated and aglycone forms (AHLUWALIA et. al., 1953). Genistin and daidzin are the main isoflavones, and they constitute 50 to 90% of the flavonoids present in soy flour (ELDRIDGE, 1982; FUKUTAKE, 1996).

Its plan chemical structures may be represented as follows:

| Plan molecular structure | Isoflavane | R1 | R2 | R3 | R4 |
|---|---|---|---|---|---|
| 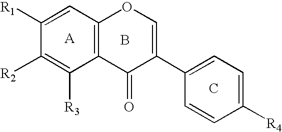 | Daidzin Daidzein Glycitin Glycitein Genistin Genistein | O-glucosyl OH O-glucosyl OH O-glucosyl OH | H H OCH3 OCH3 H H | H H H H OH OH | OH OH OH OH OH OH |

In several experimental models, isoflavones have been related to the growth-inhibitory effect against cancer cell lines, reduction of cholesterol, or even, the inhibition of bone re-absorption.

“In vitro’ studies have shown that aglycone isoflavones show greater antioxidant activities when compared to other conjugated forms (PARK et. al., 2001), as well as the most effective against cancer cells (OHR, 2002). Clinical studies, in human, indicate that aglycone isoflavones are more absorbed easily than glucosylated forms (PARK et. al, 2001; SETCHELL & CASSIDY, 1999).

Considering that certain technological process involving soy, are commonly used in industries were developed without much precautions in relation to the loss of the final product obtained from soy grains, several studies have shown that changes on the profile of several soy components may be a result of this lack of precautions as the prolonged and/or inadequate storage, which may affect both the protein functionality and the its final quantity of several components important to human beings, such as isoflavones. However, the soy grain processing produces a large amount of new soy based products, which provides a great variety of healthy products available to human diet. The great interest for soy is due to, in its great majority, to the strong presence of isoflavone in soy grains and in some soy based products.

Nevertheless, there are few efforts in the sense to preserve isoflavones content in several soy processed products. There is an urgent need to assess isoflavone loss observed in some industrial processes involving the soybean grains processing.

This processing is mainly performed to extract soy comestible oil to produce soy bran, being this product closely associated to animal ration production and for soy protein extraction, whether by the production of concentrated or isolated soy protein.

Thus, having in mind the loss of isoflavones during the process of soy-food transformation and considering the purpose of overcome disadvantages existent in technical status, it is important to develop a method for the recovery of isoflavones from sub-products of processing industry of soy protein and use them, among others, in the production of functional food.

### BRIEF DESCRIPTION OF THE INVENTION

The present invention relates to a process for the recovery of isoflavones, especially conjugated on glucosylated, acetylated and malonate forms. This process consists in promoting the recovery of different isomeric forms of isoflavones, by chromatographic adsorption techniques, convert them into the glucosylated form and a consequent transformation of glucosylated isoflavones into their respective aglycones, from the fermentative process using the culture mean containing daidzin, glycitin and genistin, by microbial transformation with fungus of the specie Aspergillus spp. which are not aphlatoxins producers, isolated from food matrix. The transformation also includes the use of hydrolytic enzymes produced by these microorganisms, in the conversion of glycosidic bonds into aglycone phenolic forms.

The present process enables the obtainment of isoflavones, to produce functional food, and/or into the form of ingredient and additive, rising in this way its nutraceutic, therapeutic appeal and commercial interest.

There has also been proposed the transformation of glucosylated isoflavone into aglycones through the action of beta-glucosidase, lactose-phlorizin-hidrolase, enzyme able to produce aglycone isoflavones, in satisfactory amounts, under controlled rectional conditions. According to the present study performed, a proper condition to produce aglycone isoflavones by Aspergillus oryzae ATCC 22786, depends on the temperature of 25-60° C.; under shaking of 100-300 rpm; rectional time of 10 minutes to 3 hours; enzymatic activity of at least 0.1 IU/mL, with starting pH of 4.5-5.0, being the production obtained from total aglycone isoflavones around 3.0 mg per gram of soy grain.

### DETAILED DESCRIPTION OF THE INVENTION

Some examples of recovery and production of isoflavone mixture are listed bellow, including the production of aglycone isoflavones, by a fermentative via with Aspergillus oryzae, using a solution of glucosylated isoflavones as substrate.

### Example 1

Obtainment process of conjugated soy isoflavones: 1000 g of soy were ground and the obtained soy flour was treated with 10,000 mL of n-hexan at 25° C. for 30 minutes and centrifuged at 10,000 rpm for 20 minutes. The residue was dried at environmental temperature and isoflavones were extracted with 10,000 mL of 60% ethanol solution at environmental temperature for 20 minutes. After this, the material was centrifuged at 10,000 rpm for 20 minutes and the supernatant was analyzed by HPLC (High Performance Liquid Chromatography) equipped with a detector of photodiodes arrangement.

### Example 2

Process for the obtainment of conjugated isoflavones of soy concentrated protein: 50 g of soy were ground and the soy flour obtained was treated with 10,000 mL of n-hexan at 25° C. for 30 minutes and was centrifuged at 10,000 rpm for 20 minutes. The residue was dried at environmental temperature and was dispersed into 1,000 mL of water at pH 4.5, adjusted with hydrochloric acid 6N, and centrifuged at 1,000 rpm for 20 minutes. The precipitated was lyophilized and is therefore the protean portion, called soy concentrated protein. The serum obtained (supernatant) containing different isoflavone forms, was collected and used for the extraction of isoflavones with 60% ethanol solution at environmental temperature for 20 minutes, after adequate lyophilization. After this process, the material was centrifuged at 10,000 rpm for 20 minutes and the supernatant was analyzed by HPLC equipped with a detector of photodiodes arrangement.

### Example 3

Process for the obtainment of conjugate isoflavones of isolated soy protein: 50 g of soy were ground and the flour obtained was treated with 10,000 mL of n-hexane at 25° C. for 30 minutes and were centrifuged at 10,000 rpm for 20 minutes. The residue was dried at environmental temperature and was submitted to alkaline extraction with 1,000 mL of sodium hydroxide solution at pH 9.0 for 45 minutes and at 55° C., was centrifuged at 10,000 rpm for 20 minutes. The precipitated obtained was lyophilized and it corresponds to the insoluble portion of residues. The soluble fraction was submitted to protein precipitation, with a solution with pH 4.5, and was centrifuged at 10,000 rpm for 20 minutes. The precipitate corresponds to the protean portion and was called isolated soy protein. The serum obtained (supernatant) containing different forms of isoflavones, was collected and used for the extraction of isoflavones with a 60% ethanol solution at environmental temperature for 20 minutes, after adequate lyophilization. After this, the material was centrifuges at 10,000 rpm for 20 minutes and the supernatant was analyzed by HPLC equipped with a detector of photodiodes arrangement.

### Example 4

### Process for the Obtainment of Enzyme from Aspergillus oryzae

This microorganism was obtained through the genetic improvement by conventional process of mutant induction with ultraviolet light. The production of beta-glucosidase was performed inoculating the microorganism in a semi-solid medium, using conic flasks of 500 mL containing 20 g of soy grains bran or 50% humid wheat, incubating it into the stove at 30° C. for 72 hours. After this fermentation period, 100 mL of distilled water was added, and the maceration of solid medium was performed under shaking for 1 hour. After this, a filtration was performed and the enzymatic extract was collected. Ethanol was added to the enzymatic extract, in the rate of 70% of 96% ethanol for 30% of extract, left in a refrigerated environment during 15 minutes. In continuation, a centrifugation was performed to separate the enzymatic precipitate, which was dried in a glass plate under an air flow.

### Example 5

Transformation process of glucosylated isoflavone into aglycones: the process was performed through the addition of 100 mL of enzymatic extract (0.5 IU/mL) of Aspergillus oryzae, in 1,000 mL of the residual protean serum of the soy processing containing isoflavones. The residual serum of the soy protein supplemented with the enzymes was incubated for 1 hour at 40° C., for the conversion of glucosylated isoflavones into aglycones. The residual serum can be then, percolated by adsorption resins for the removal of impurities and residues, such as Amberlite XAD-2 and Amberlite XAD-16 resins. The eluted obtained may be concentrated by evaporation at low pressure, and employed in the production and enrichment of functional food.

The description above of the present invention was showed with the purpose of illustration and description. Besides, the description does not intended to limit the invention to the form exposed herein. Consequently, variations and modification compatible to the aforesaid and the capacity or awareness of relevant technique, are within the scope of the present invention.

The modalities described above intend to better explain the common manners for the execution of the invention and to allow technicians of this field, to use it, in such, or other modalities and with several necessary modifications by specific applications or uses of the present invention. The intention is that the present invention includes all its modifications and variations within the scope described in the report and in attached claims.

1.-58. (canceled)

59. Process for the obtainment of enzymes from Aspergillus oryzae, wherein the microorganism used is obtained by genetic improvement through conventional processes of mutant induction with radiation and includes the following steps:
a) inoculation of microorganism;
b) fermentating;
c) maceration of medium;
d) shaking;
e) filtering, with collection of enzymatic extraction;
f) centrifuging; and
g) drying.

60. Process for the obtainment of enzymes from Aspergillus oryzae according to claim 59 wherein the main enzyme obtained is beta-glucosidase.

61. Process for the obtainment of enzymes from Aspergillus oryzae according to claim 59 wherein the inoculation of the microorganism of step a) is preferably performed in a semi-solid medium with humid grains bran, as soy and wheat.

62. Process for the obtainment of enzymes from Aspergillus oryzae according to claim 59 wherein the fermentation of the microorganism of step b) is preferably performed in a stove at 20 to 40° C. for 2 to 4 days.

63. Process for the obtainment of enzymes from Aspergillus oryzae according to claim 59 wherein the meceration of the solid medium of step c) is performed after the addition of distilled water and after shaking for about 30 minutes to 2 hours.

64. Process for the obtainment of enzymes from Aspergillus oryzae according to claim 59 wherein the precipitation of the enzymatic extract of step t) is preferably performed through the addition of ethanol at the approximate rate of 50 to 80% of 96% ethanol, stored in refrigerated environment for 5 to 30 minutes.

65. Process for the obtainment of enzymes from Aspergillus oryzae according to claim 59 wherein the enzymatic precipitate separated in step g) is dry, preferably in a glass place under air circulation (step h)).

66. Process for the transformation of glucosylated isoflavones into aglycones from Aspergillus oryzae wherein the use of the enzymatic extract obtained according to the steps described in claim 59 includes the following steps:
a) adding enzymatic extract to the protean serum;
b) conversion of glucosylated isoflavones into aglycones;
c) filtering; and
d) concentrating.

67. Process for the transformation of glucosylated isoflavones into aglycones from Aspergillus oryzae according to claim 66 wherein step a) adds about 0.1 to 1.0 IU/ml of enzymatic extraction to the protean serum.

68. Process for the transformation of glucosylated isoflavones into aglycones from Aspergillus oryzae according to claim 66 wherein protean serum used in step a) is obtained from the liquid residues of soy protean concentrate and soy protein isolate production (soy molasses).

69. Process for the transformation of glucosylated isoflavones into aglycones from Aspergillus oryzae according to claim 68 wherein the liquid residues of soy protean concentrate are obtained by the following steps:
a) providing soybean;
b) grinding;
c) defatting;
d) precipitation of Soy Protein at pH 4.5;
e) centrifuging; and
f) liquid residue+soy protein concentrate.

70. Process for the transformation of glucosylated isoflavones into aglycones from Aspergillus oryzae according to claim 68 wherein the liquid residues of soy protein isolate is obtained by the following steps:
a) producing soybean;
b) grinding;
c) defatting;
d) solubilization of Protein at pH 9.0;
e) centrifuging;
f) solid residue (okara)+liquid protein solution;
g) liquid protein solution;
h) acidification at pH 4.5;
i) precipitation of proteins;
j) centrifugation; and
k) liquid residue+soy protein isolate.

71. Process for the transformation of glucosylated isoflavones into aglycones from Aspergillus oryzae according to claim 66 wherein the conversion of glucosylated isoflavones into aglycones described in step b) is performed through the incubation for the period of 30 minutes to 2 hours at 25 to 50° C.

72. Process for the transformation of glucosylated isoflavones into aglycones from Aspergillus oryzae according to claim 66 wherein the filtering to residual serum obtained in step b) is performed preferably through the percolation process to remove impurities and residues.

73. Process for the transformation of glucosylated isoflavones into aglycones from Aspergillus oryzae according to claim 66 using adsorption resins in the percolation of the residual serum.

74. Process for the transformation of glucosylated isoflavones into aglycones from Aspergillus oryzae according to claim 66 wherein the adsorption resins used in the percolation of residual serum are Amberlite XAD-2 and Amberlite XAD-16 or any resin or other equivalent material.

75. Process for the transformation of glucosylated isoflavones into aglycones from Aspergillus oryzae according to claim 66 wherein the elution obtained from step c) is concentrated by evaporation at a low pressure (step d)).

76. Biologically pure culture of microorganism Aspergillus oryzae with a storage number ATCC 22786 (RIB 430), obtained by genetic improvement by conventional processes on mutant induction, increasing its enzymatic production.

77. Biologically pure culture of microorganism Aspergillus oryzae according to claim 76 wherein the conventional process of genetic improvement is the exposition of ultraviolet light.

78. Biologically pure culture of microorganism Aspergillus oryzae according to claim 77 wherein the enzymatic production is increase of beta glucosidase production in the enzymatic extract of the microorganism.

79. Biologically pure culture of microorganism Aspergillus oryzae according to claim 76, which produces an enzyme which catalyzes the reaction of glucosylated, acetylated and malonated isoflavones into aglycones isoflavones.

80. Biologically pure culture of microorganism Aspergillus oryzae according to claim 76, wherein the microorganism is derived from a more gastronomically acceptable strain.

81. Mutant microorganism Aspergillus oryzae with storage number ATCC 22786 (RIB 430), showing a greater final production of beta-glucosidase.

82. The Mutant of claim 81 which is more gastronomic acceptable.

83. The Mutant of claim 81, which can produce an enzyme which catalyzes the reaction of glucosylated, acetylated and malonated isoflavones into aglycone isoflavones.

84. Mutant fungus which is a more gastronomic acceptable strain able to produce an enzyme which catalyzes the reaction of glucosylated acetylated and malonated isoflavones into aglycone isoflavones.

85. Mutant fungus, according to claim 84 wherein the fungus is Aspergillus oryzae with storage number ATCC 22786 (RIB 430) showing a great final production of beta-glucosidase.

86. Food composition containing aglycone isoflavones transformed from soy glucosylated isoflavones through the enzymatic processing of the microorganism Aspergillus oryzae and food additives.

87. Food composition according to claim 86 wherein the soy used is in the form of flour, ground, concentrated and any other form which show aglycone and glucosylated isoflavones.

88. Drug composition confining aglycone isoflavones transformed from soy glucosylated isoflavone through the enzymatic processing of the microorganism Aspergillus oryzae and vehicles pharmaceutically acceptable.

89. Drug composition according to claim 88 wherein the soy used is in the form of ground, concentrated flour and any other form which has aglycones and glycosylated isoflavones.
